(19) United States
(12) Patent Application Publication
(10) Pub. No.: US 2025/0375598 A1
(43) Pub. Date: Dec. 11, 2025

(54) SYSTEM FOR HEALTH QUALITY/PERFORMANCE IMPROVEMENT USING NASAL DILATORS AND RELATED METHODS

(71) Applicant: Breathewave Technologies Inc., Edgewater, NJ (US)

(72) Inventors: David J. Axelrad, Closter, NJ (US); Michael A. Linn, Ridgewood, NJ (US); Geoffrey P. Waite, Campton, NH (US); Christopher A. Beaudreau, Parker, CO (US)

(21) Appl. No.: 19/228,320

(22) Filed: Jun. 4, 2025

Related U.S. Application Data
(60) Provisional application No. 63/656,450, filed on Jun. 5, 2024.

(51) Int. Cl. A61M 29/00; A61B 5/00

(52) U.S. Cl. CPC A61M 29/00; A61B 5/4848

(57) ABSTRACT

A method for enhancing health quality and performance is disclosed. The method involves providing multiple sets of nasal dilators, each set having a distinct configuration. The dilator set being used is then identified. The health quality/performance of the user using the identified set of nasal dilators is then monitored using a health quality/performance monitoring and feedback system. Additionally, the health quality/performance monitoring and feedback system provides feedback on the user's health quality/performance based on the identified set of nasal dilators. This method allows for personalized and targeted improvement of health quality and performance by optimizing the use of nasal dilators.

### CROSS-REFERENCE TO RELATED APPLICATION(S)

This application claims priority to, and the benefit of, co-pending U.S. Provisional Application 63/656,450, filed Jun. 5, 2024, for all subject matter common to both applications. The disclosure of said provisional application is hereby incorporated by reference in its entirety.

### FIELD OF THE INVENTION

The present invention pertains to an integrated system that combines the use of nasal dilators as a breathing improvement device with a health quality/performance monitoring and feedback system to monitor performance while using nasal dilators and collect data as to what features or characteristics of nasal dilators provide improved benefit for a user.

### BACKGROUND

The field of breathing quality improvement, particularly sleep quality improvement, including snoring reduction, has seen significant advancements in recent years. One area of focus has been on the development of devices designed to improve breathing during sleep or other activities, such as nasal dilators. These devices are designed to physically open the nasal passages, thereby reducing the resistance to airflow and potentially improving athletic performance and/or sleep quality. However, the effectiveness of these devices can vary greatly from person to person, depending on factors such as the individual's nasal anatomy and the specific design of the dilator.

In addition to these dilator devices, there has been a growing interest in the use of health quality/performance monitoring devices and/or applications such as health trackers or sleep quality monitors. Applications may run on personal devices such as cell phones. These health quality/performance devices and/or applications can provide valuable data on various aspects of user health or performance during activities or during sleeping, which can be used to assess the overall sleep or performance quality. However, there is a lack of integration between these two types of devices, specifically between the data from trackers and monitors and the use of nasal dilators. Such lack of integration limits their potential effectiveness.

Furthermore, there is a lack of data on the relationship between the design of nasal dilators and their effectiveness in improving breathing and health quality/performance. This lack of data makes it challenging to optimize the design of these devices for both the general population and individual users. Additionally, there is a need for a more systematic approach to selecting nasal dilators, as the current method is largely based on subjective trial and error.

In summary, while significant advancements have been made in the field of breathing-based health quality and performance improvement, several challenges remain to be addressed. These include the need for improved technological integration between different types of devices, the need for more data on the relationship between dilator device design and effectiveness, and the need for a more systematic approach to dilator device design and/or selection.

### SUMMARY

The present invention relates to an integrated system in which the use of one or more nasal dilators as breathing improvement devices is combined with a health quality/performance monitoring and feedback system to provide a) feedback to the user on device efficacy and recommendations for alternative device shapes or sizes as well as b) providing feedback, including through the agglomeration of data from multiple users, to designers and providers of nasal dilators. Additional device designs and recommendations can be made for the user and/or the broader population based on this feedback. This further allows the provided improvement to be iterative with each generation or selection of device, with each one providing data used in the design or selection of the next generation of device.

### BRIEF DESCRIPTION OF THE FIGURES

These and other characteristics of the present invention will be more fully understood by reference to the following detailed description in conjunction with the attached drawings, in which:

FIG. 1 is a system diagram showing the various components involved in the present invention and their interactions in accordance with certain embodiments of the present invention;

FIG. 2 is a diagram of a kit providing two or more nasal dilators having different configurations in accordance with certain embodiments of the present invention;

FIG. 3 is a close-up of the nasal dilators showing the identifiers of the nasal dilators in accordance with certain embodiments of the present invention;

FIG. 4 is an exemplary user interface provided by a health quality/performance monitoring and feedback system in accordance with certain embodiments of the present invention

FIG. 5 is an exemplary computing device that may be used in accordance with certain embodiments of the present invention;

FIG. 6 is a flow chart of a methodology for improving health quality/performance in accordance with certain embodiments of the present invention;

FIG. 7 is an example of identifying a nasal dilator based on a scan of a user's nose in accordance with certain embodiments of the present invention;

FIG. 8 is a flow chart of a methodology for using a remote computing device for the analysis of health quality/performance data in accordance with certain embodiments of the present invention; and

FIG. 9 is a flow chart of a methodology for using health quality/performance data to design and provide additional nasal dilators to a user in accordance with certain embodiments of the present invention.

### DETAILED DESCRIPTION

An illustrative embodiment of the present invention relates to a technologically integrated system in which the use of one or more nasal dilators as a breathing improvement device is combined with a health quality/performance monitoring and feedback system to provide feedback to the user on device efficacy and recommendations for alternative device shapes or sizes as well as to provide feedback including through the agglomeration of data from multiple users to the designers or providers of nasal dilators. The feedback can further inform the design of additional devices. These additional devices can be provided for the user and for the broader population to try. Thus, the provided improvement can be iterative with each generation of nasal dilator, providing data used in the design of the next generation of nasal dilators.

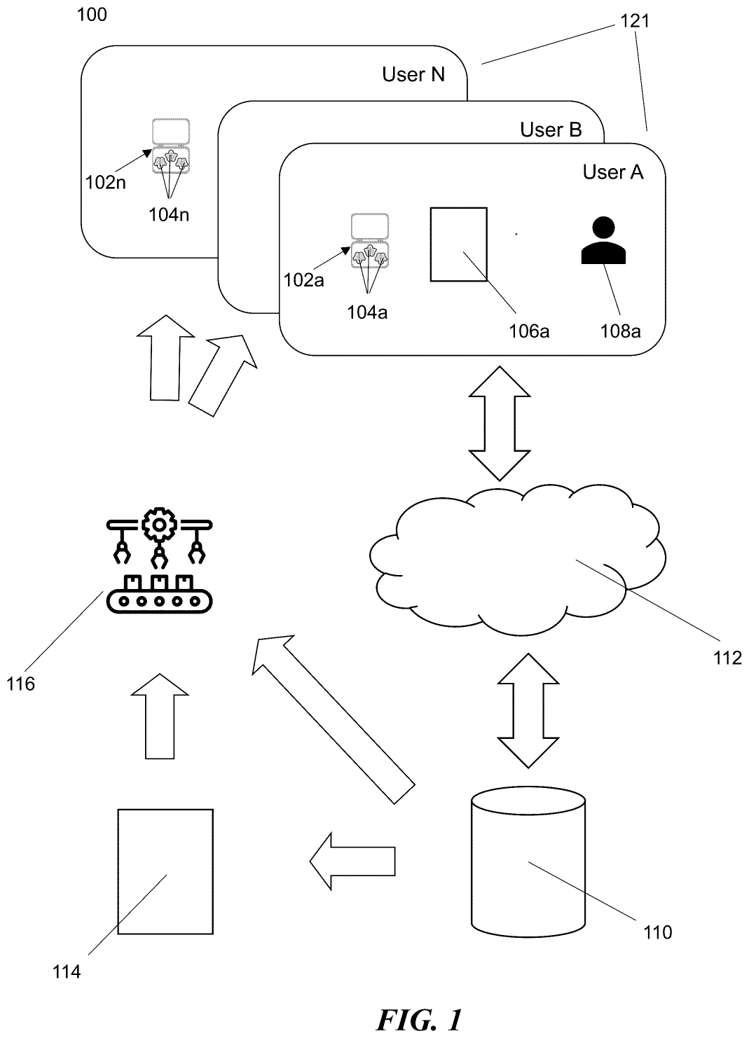

FIG. 1 through FIG. 9, wherein like parts are designated by like reference numerals throughout, illustrate an example embodiment or embodiments of an integrated system for sleep quality improvement using nasal dilators and related methods, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, type of elements or materials, computing architecture, and algorithms, in a manner still in keeping with the spirit and scope of the present invention.

FIG. 1 is a system diagram 100 illustrating the system and its environment. Here, this comprises providing at least two sets of nasal dilators 104 to a user 108, wherein each set of nasal dilators 104 has a different configuration. The two or more nasal dilators 104 can be provided individually and/or sequentially over time, for example, purchased or ordered by the user 108 based on the user's 108 quality/performance needs or based on feedback provided to the user 108. In other embodiments the at least two nasal dilators are provided as part of a sample/testing kit 102 as shown here.

In addition, a health quality/performance monitoring and feedback system 106 is provided for use by the user 108 to monitor health quality and performance and receive feedback, including recommendations to influence health quality and performance. The health quality/performance monitoring and feedback system 106 can comprise software, hardware, or a combination of both, which may be embodied in one or more discrete devices. Examples of such quality monitoring and feedback systems that are well known include acoustic monitoring of sleep breathing, e.g., using a cellphone, and body-worn fitness systems monitoring heart rate, SpO2, breathing rate, skin or body impedance, blood pressure and/or several other factors.

As shown here, there can be multiple deployments 121 of the system. Here, there are N deployments 121, where for each deployment, the user 108a-108n is provided with a kit 102a-102n having the at least two or more nasal dilators 104a-104n and a health quality/performance monitoring and feedback system 106a-106n of the present invention.

In certain embodiments, the health quality/performance monitoring and feedback system 106 is further in communication with a remote computing device 110, such as a server or cloud computing device over a network 112, such as the Internet or the World Wide Web. The use of a remote computing device allows for the collection and aggregation of data from multiple users 108, which can be used to provide feedback to the users and/or the designer 114 and/or provider/manufacturer 116 of the nasal dilators. The one or more of the remote computing device 110, designer 114 and provider/manufacturer 116 are shown as separate entities, and it should be understood that they can be the same or part of the same entity wherein the functionality provided individually by the remote computing device 110, designer 114 and provider/manufacturer 116 may be combined together with or omitted from one or more of the remote computing device 110, designer 114 and provider/manufacturer 116.

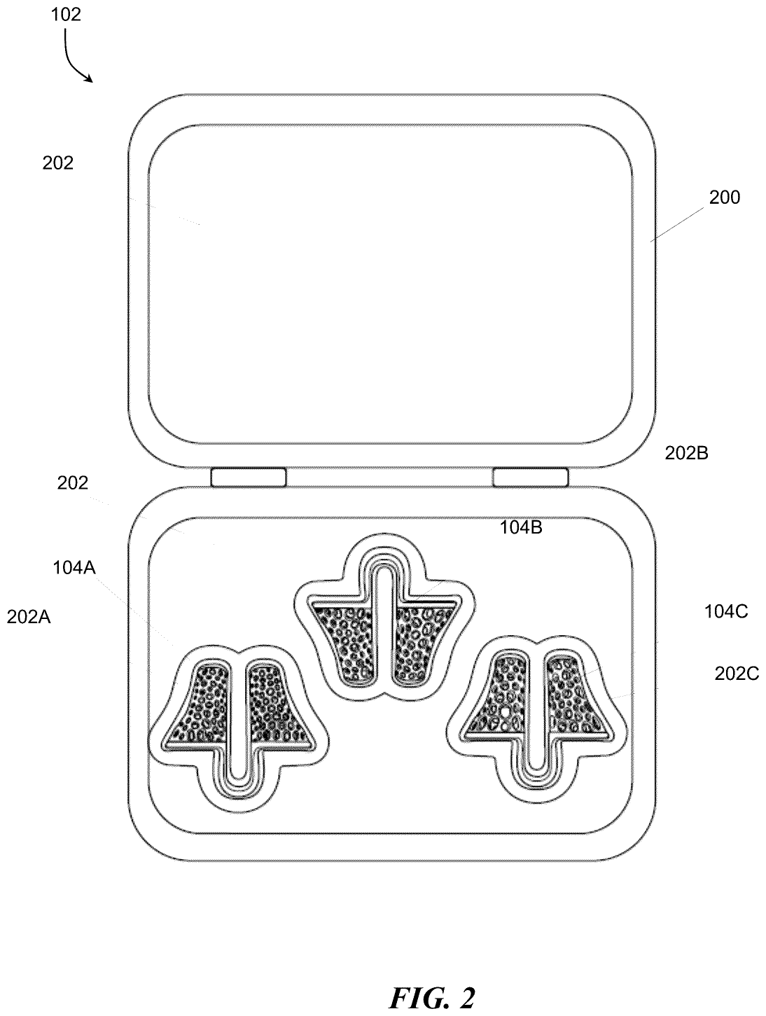

FIG. 2 shows the sample/testing kit 102 from FIG. 1. Here the kit 102 includes a storage case 200 sized, dimensioned, and configured for storing the at least two sets of nasal dilators 104 selected to provide testing baselines for the user 108. Here the storage case 200 is a clamshell design with an interior cavity 202 to hold the sets of dilators 104A, 104B, 104C, in their individual cavities 202A, 202B, 202C, though other configurations known to those of skill in the art are considered to fall within the scope of the present invention. The storage case 200 individual cavities 202A, 202B, 202C, optionally may include identifiers formed of symbols, colors, and/or shapes, such as to identify the individual set of dilators contained therein.

Each of the at least two sets of nasal dilators 104 has a configuration different from the other provided sets. In one such embodiment, the nasal dilators 104 are deliberately produced with variations around a declared size and/or shape profile (“fit perturbations”), each of which is uniquely identified during the collection of the data by the health quality/performance monitoring and feedback system 106. The collection of data for such fit perturbations may provide additional points within the dataset that can be used to produce more efficacious designs, including for the training of design algorithms used by the designer(s) 114 to translate from nose profiles or nose profile classes to optimized dilator profiles. These optimized dilator profiles can then be used to manufacture or otherwise produce new dilators 104 by the provider/manufacturer 116. These new dilators, in turn, can be provided to the user(s) 108 for use with further health quality/performance monitoring and feedback.

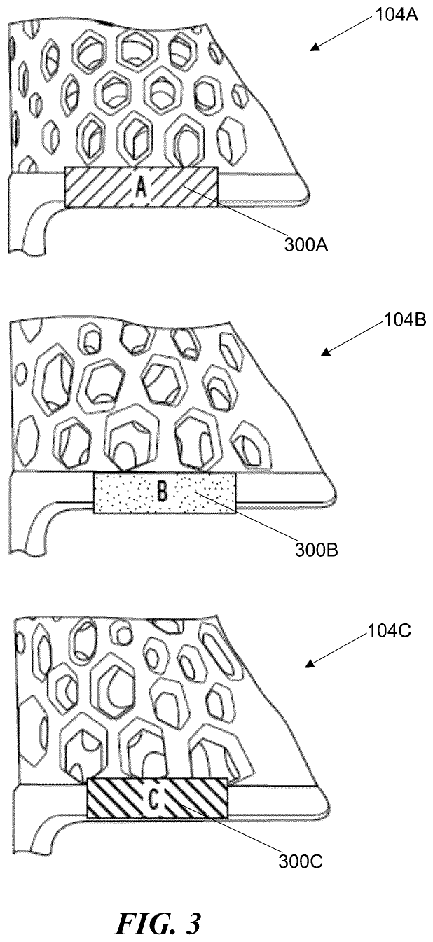

In accordance with some embodiments, the at least two sets of nasal dilators 102 further comprise unique identifiers 300 A, B, and C to distinguish each set from another. In some such embodiments, the identifiers 300 A, B, C comprise one or more of symbols, colors, shapes, and electronically readable tags such as RFID, NFC, or a barcode. An example of this can be seen in FIG. 3, where a close-up view of a section of the sets of nasal dilators 104A, 104B, and 104C is shown, with identifiers 300A, 300B, and 300C. In this case, a color and letter designation serve as the identifiers. This identifier 300 A, B, C can be used to identify and register which nasal dilator is being used by the user 108.

While the examples provided here focus on internal nasal dilators 104, it should be understood that the teachings of the present invention can also be used in conjunction with external nasal dilators.

The system 100, via the health quality/performance monitoring and feedback system 106 or remote computing device 110, is configured to receive identification of which set of nasal dilators of the at least two sets of nasal dilators is used by the user, monitor the health quality/performance of the user using the identified nasal dilator, and provide feedback based on the monitored health quality/performance of the user using the identified nasal dilator.

The health quality/performance monitoring and feedback system 106 can include any device and/or software configured to monitor the health quality/performance of the user 108. Such monitoring and feedback systems exist and include both professional-grade and consumer-grade systems; two examples of useful consumer-grade systems are acoustic monitoring for measuring snoring levels and other audible sleep disturbances, often deployed on a personal device such as a smartphone, and physiological tracking using a fitness or sleep tracker with combinations of sensors to detect motion, pulse, blood pressure, blood oxygen levels, breathing rate, skin temperature, resistivity, and other physiological parameters. Both classes of systems can be used to drive sophisticated learning algorithms that determine the sleep stage architecture, level, and physiological impact of snoring and loud breathing, as well as other physiological and environmental factors.

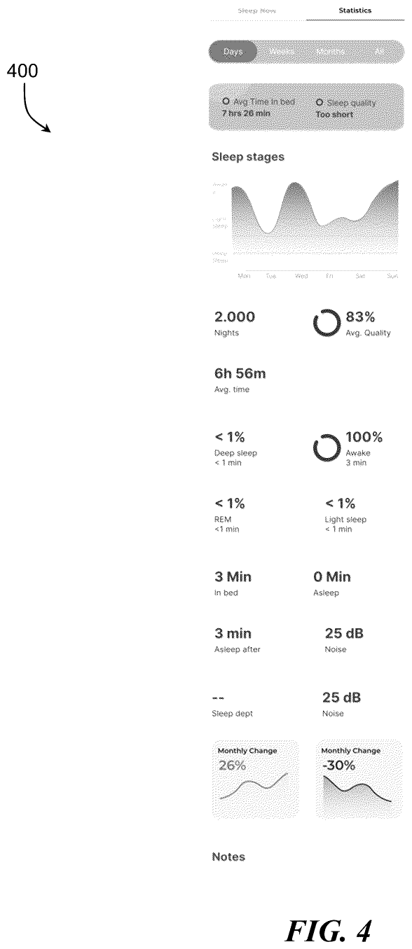

The health quality/performance monitoring and feedback system 106 can be a dedicated device having the necessary sensors (microphones, cameras, biometric sensors, etc.) for monitoring health quality and/or performance, as well as, optionally, the hardware and/or software to provide additional functionality of identifying which set of nasal dilators of the at least two sets of nasal dilators is used by the user and providing feedback based on the monitored health quality/performance of the user using the identified nasal dilator. This includes a processor for processing data collected by the sensors and a user interface including an input mechanism for receiving input provided by the user 108 and a display mechanism for displaying data to the user 108. In accordance with some embodiments, the health quality/performance monitoring and feedback system 106 can be a personal computing device of the user 108, such as a mobile device, running software that configures the device to operate as a health quality/performance monitoring and feedback system. The software may make use of sensors incorporated into or connected to the personal computing device, such as microphones, speakers, home automation devices, smart speakers, smart watches, biometric sensors, fitness trackers, etc., to collect health quality and/or performance data for the user. An example of a user interface 400 displayed to a user 108 by the health quality/performance monitoring and feedback system 106 can be seen in FIG. 4.

In certain embodiments, the health quality/performance monitoring and feedback system 106 may transmit or otherwise provide collected health quality and/or performance data for the user 108 to a remote computing device 110, such as a server or cloud computing device, for storage and/or processing of the data. The received data can also be analyzed using artificial intelligence (AI), including machine learning, to determine patterns, commonalities, correlations, and indicators in the data. For example, data can be collected and used to build and extend a multi-user 108 labeled dataset of dilator 104 characteristics and health quality/performance outcomes. Such a dataset is used to determine and train design algorithms, including but not limited to using machine learning techniques, running on the remote computing device 110, designer 114, and/or provider/manufacturer 116 to derive and enhance models of the interaction of physiological factors and the design of an optimum internal nasal dilator for those physiological factors, including the optimum mathematical transformation from a nose or nostril shape to a nasal dilator shape and the circumferential force profile determining how much dilation is applied in different directions. The use of this integrated system allows both the user (via the provided feedback) to select more efficacious dilators 104, and the designer 114 and/or provider/manufacturer 116 to fabricate more efficacious devices for the user 108 and/or other users 108 and for specific cohorts of users 108 collectively, such cohorts which may have been delineated by analysis of the same dataset.

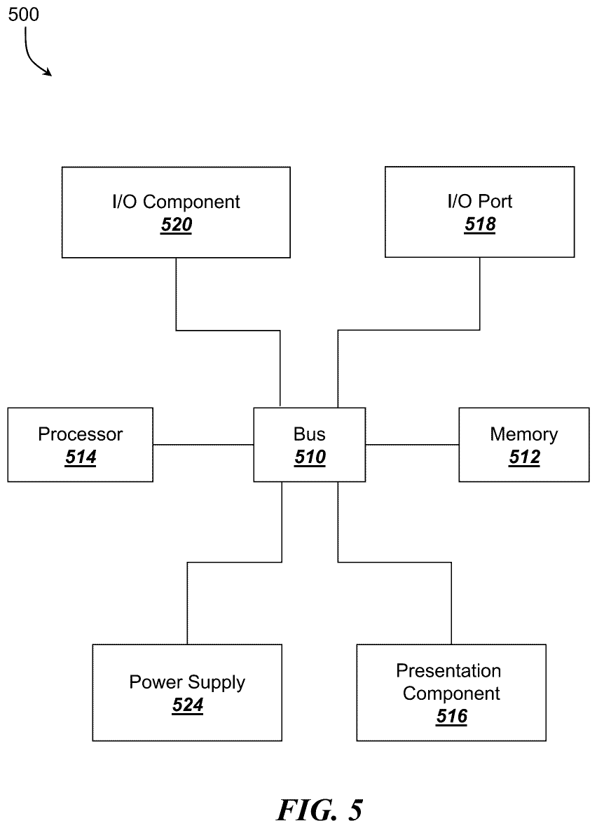

Suitable computing devices can be used to implement health quality/performance monitoring and feedback system 106, remote computing device 110, functionality of the designer 114, and/or provider/manufacturer 116. One illustrative example of such a computing device 500 is depicted in FIG. 5. The computing device 500 is merely an illustrative example of a suitable computing environment and in no way limits the scope of the present invention. A “computing device,” as represented by FIG. 5, can include a “workstation,” a “server,” a “laptop,” a “desktop,” a “hand-held device,” a “mobile device,” a “tablet computer,” a “wearable device” or other computing devices, as would be understood by those of skill in the art. Given that the computing device 500 is depicted for illustrative purposes, embodiments of the present invention may utilize any number of computing devices 500 in any number of different ways to implement a single embodiment of the present invention. Accordingly, embodiments of the present invention are not limited to a single computing device 500, as would be appreciated by one with skill in the art, nor are they limited to a single type of implementation or configuration of the example computing device 500.

The computing device 500 can include a bus 510 that can be coupled to one or more of the following illustrative components, directly or indirectly: a memory 512, one or more processors 514, one or more presentation components 516, input/output ports 518, input/output components 520, and a power supply 524. One of skill in the art will appreciate that the bus 510 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, FIG. 5 is merely illustrative of an exemplary computing device that can be used to implement one or more embodiments of the present invention, and in no way limits the invention.

The computing device 500 can include or interact with various computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 500.

The memory 512 can include computer storage media in the form of volatile and/or non-volatile memory. The memory 512 may be removable, non-removable, or any combination thereof. Exemplary hardware devices include devices such as hard drives, solid-state memory, optical disc drives, and the like. The computing device 500 can include one or more processors that read data from components such as the memory 512 and the various I/O components 516. Presentation component(s) 516 present data indications to a user or other device. Exemplary presentation components include a display device, a speaker, a printing component, a vibrating component, etc.

The I/O ports 518 can enable the computing device 500 to be logically coupled to other devices, such as I/O components 520. Some of the I/O components 520 can be built into the computing device 500. Examples of such I/O components 520 include a microphone, joystick, recording device, game pad, satellite dish, scanner, printer, wireless device, networking device, and the like.

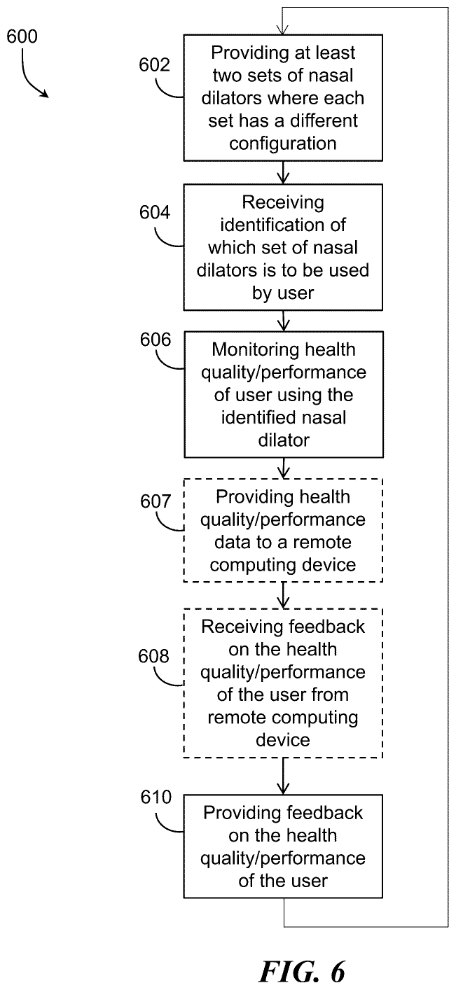

A methodology for the operations of the system 100 is set forth in the flow chart 600 of FIG. 6. Here, the first step comprises the user being provided with or otherwise obtaining at least two sets of nasal dilators 104, wherein each set of nasal dilators has a different configuration (Step 602). The next step is receiving identification of which set of nasal dilators 104 is used or to be used by the user 108 (Step 604). The third step is monitoring, using the health quality/performance monitoring and feedback system 106, the health quality/performance of the user 108 while using the identified set of nasal dilators 104 (Step 606). In certain embodiments, the one or more optional steps of: providing health quality/performance data to a remote computing device 110 (Step 607) and receiving feedback from the remote computing device 110 on the health quality/performance of the user 108 (Step 608) can be performed. The final step set forth in the flow chart 600 is providing, using the health quality/performance monitoring and feedback system 106, feedback on the health quality/performance of the user 108 using the identified set of nasal dilators 104 (Step 610).

The two or more sets of nasal dilators 104 can be provided individually, simultaneously, and/or sequentially over time. For example, dilators may be purchased or ordered by the user 108 based on the quality/performance needs of the user 108 or based on feedback provided to the user 108. In other embodiments, the two or more dilators 104 are provided together as part of the kit 102 (Step 602). Each nasal dilator 104 may be uniquely identified using an identifier 300.

The identifier 300 can then be provided to the health quality/performance monitoring and feedback system 106 or the remote computing device 110 to identify which nasal dilator is used or to be used (Step 604). In certain embodiments, the health quality/performance monitoring and feedback system 106 or remote computing device 110 receives the identification of the nasal dilators 104 used or to be used from the user 108. In some such embodiments, this may involve receiving an input from the user on a user interface of the health quality/performance monitoring and feedback system 106 or remote computing device 110. In still further embodiments, this may involve scanning the unique identifier 300 of the set of nasal dilators 104 to be used. In one embodiment of this system, automatic registration is achieved via the identifier 300 on a specific nasal dilator 104 to allow a scanning mechanism, for example, a camera or a Near Field Communication (NFC) reader on the health quality/performance monitoring and feedback system 106 to identify the specific nasal dilator 104 shape variant in use and correlate this to the health quality/performance monitoring data collected.

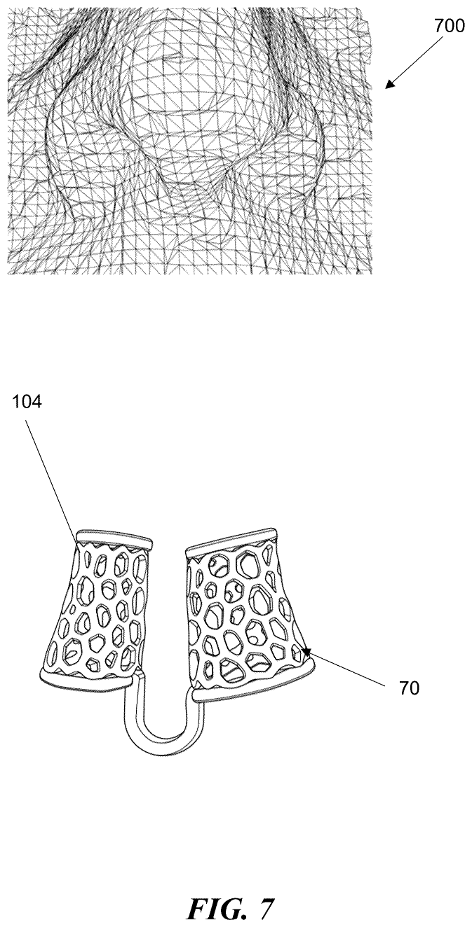

In some example embodiments, identifying which set of nasal dilators 104 of the at least two sets of nasal dilators 104 is used or to be used by the user (Step 604) comprises providing, by the health quality/performance monitoring and feedback system 106, a recommendation of a set of nasal dilators 104 of the at least two sets of nasal dilators. This can be done by providing the user 108 with a series of questions, via the user interface of the health quality/performance monitoring and feedback system 106 to help identify issues to be addressed with a nasal dilator 104 to determine which nasal dilator 104 the user 108 should use. In another embodiment, scans of the user's 108 nose can be provided to the health quality/performance monitoring and feedback system 106, for example by using a camera of the health quality/performance monitoring and feedback system 106, to determine the appropriate nasal dilator 104. An example of this can be seen in FIG. 7. Here a scan 700 of the users nose is obtained, a nasal dilator is matched to the scan, and the appropriate nasal dilator 104 is identified 702 to the user. Once, the nasal dilator 104 used or to be used is identified the health quality/performance monitoring and feedback system 106 can then monitor the health quality/performance of the user 108 while using the identified nasal dilator 104 (Step 606). In some embodiments, this involves tracking the sleep quality of the user 108 while using the identified nasal dilator 104 using audio analysis. In some embodiments, other biometric measurements may alternatively or additionally be received and analyzed including heart rate, blood oxygen levels, and activity level. The health quality and/or performance data can be taken directly by the system 106 or system application or can be taken from an API with the monitoring systems 106 software platform.

Feedback on the health quality/performance of user 108 using the identified set of nasal dilators 104 can then be provided to the user 108. In some embodiments, this may involve providing the user 108 with the data collected during health quality/performance monitoring for the user 108 using the user interface of the health quality/performance monitoring and feedback system 106.

In certain embodiments, providing feedback (step 608) comprises analyzing data collected during health quality/performance monitoring (Step 606) and providing a recommendation to the user 108 based on analysis.

In some such embodiments, this involves recommending and/or providing to the user 108 one or more additional nasal dilators 104 based on the analysis of the data collected during health quality/performance monitoring (Step 606). In still further such embodiments the size, shape, and configuration of the provided one or more additional nasal dilators 104 is selected based on the analysis of the data collected during health quality/performance monitoring. In some such further embodiments, the recommended and/or provided one or more nasal dilators 104 are designed specifically for the user 108 based on the analysis of the data collected during health quality/performance monitoring.

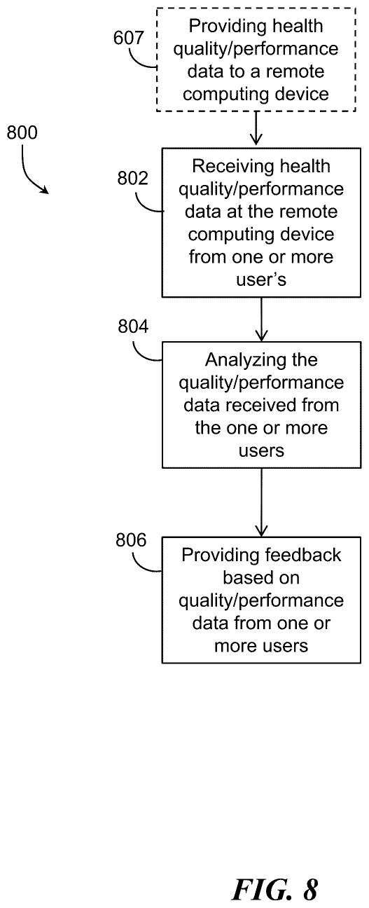

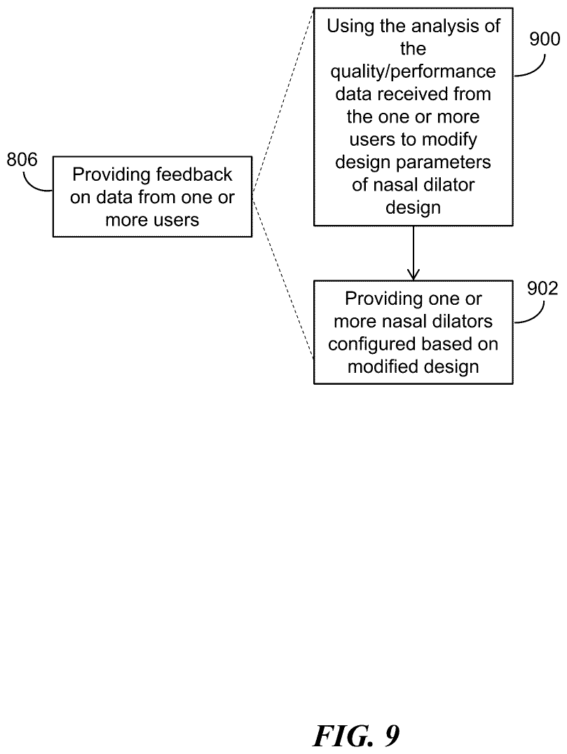

In some embodiments, the feedback provided is based on analysis by a remote computing device 110 (Step 608) that has been provided data regarding the health quality/performance of the user 108 by the health quality/performance monitoring and feedback system 106 (Step 607). FIG. 8 and FIG. 9 depict some examples of his process.

In the first step (step 802) of the process 800 of FIG. 8, the remote computing device 110 receives quality/performance data for one or more users 108 transmitted by the user(s) 108 respective health quality/performance monitoring and feedback system 106. (Step 607 of FIG. 6). The received data from the one or more users 108 can then be analyzed to determine if any correlations between a specific dilator design, features and/or characteristics, and health quality/performance for the one or more users 108 exist (step 804). In some embodiments, this analysis involves agglomeration of data from multiple users 108 to determine correlations for groups of users 108. In still other embodiments, this analysis can be performed or assisted by the use of artificial intelligence (A.I) to determine patterns, commonalities, correlations, and indicators in the data. The results of this analysis can then be provided by the remote computing device as feedback (step 806). In some embodiments, this feedback may just be the results of the analysis that are provided back to the respective health quality/performance monitoring and feedback system 106 of the one or more users 108 (Step 608 of FIG. 6). In still other embodiments, this feedback is based on the data collected from other users. In still further embodiments, the feedback can include a recommendation for a different nasal dilator or for the design of a new nasal dilator based on the analysis. This design can be based on a specific user's data, data collected from other users, and/or an amalgamation of data from multiple users. An example of this can be seen in FIG. 9.

Here the step of providing feedback (step 806) further comprises using the analysis of the received quality/performance data from the one or more users 108 to modify design parameters of a nasal dilator (Step 900) and providing one or more nasal dilators 104 configured based on the modified design (Step 902).

The modification of design parameters (step 900) can be performed based on the patterns, commonalities, correlations, and indicators in the data. determined during the analysis (step 704) of the quality/performance data for the one or more users 108. The resulting design can be modified specific to a particular user 108 or modified for use by a group of users 108. In certain embodiments, the modification of the design is performed by a designer 114 provided with the analysis from the remote computing device 110.

The providing of one or more nasal dilators 104 configured based on the modified design (Step 902) can involve providing the modified design (performed by the designer at Step 900) to a provider/manufacturer 116 who fabricates one or more dilators based on the modified design. The fabricated nasal dilators may then be provided to the one or more users 108.

Once the feedback (which may include, analysis, recommendations, and or additional nasal inserts) has been provided (step 610) the process can then be repeated with the user identifying another set of nasal dilators 104 used or to be used based on the provided feedback. In some embodiments, this can include identifying one of the newly provided one or more nasal dilators 104 used or to be used, monitoring the health quality/performance of the user 108 using the identified nasal dilator 104, and providing feedback.

The present invention provides a mechanism by which data is collected and used to build and extend a labeled dataset of dilator characteristics and quality/performance outcomes for a particular user or across many users. This may include analysis or processing using artificial intelligence (AI), including machine learning, to determine patterns, commonalities, correlations, and indicators in the data. Such a dataset can be used to determine and train design algorithms, including machine learning types, to derive and enhance models of the interaction of physiological factors with the design of an optimum internal nasal dilator for those physiological factors, including the optimum transformation from a nose or nostril shape to a nasal dilator shape and the circumferential force profile determining how much dilation is applied in different directions. This process may also be iterative, where the monitored data for the quality/performance of each identified nasal dilator, the analysis performed, and feedback provided is built upon the monitored data for the quality/performance of each identified dilator, the analysis performed, and feedback provided previously. This in turn allows for the continuous improvement of the health quality/performance provided by the nasal insert 104 to the user 108 or group of users 108.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may exist in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extent or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may, in some instances, depend on the specific context. However, in general, the nearness of completion will be to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art given the foregoing description. Accordingly, this description is to be construed as illustrative only and is to teach those skilled in the art the best mode for carrying out the present invention. Details of the structure may vary substantially without departing from the spirit of the present invention, and exclusive use of all modifications that come within the scope of the appended claims is reserved. Within this specification, embodiments have been described in a way that enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A method of improving health quality/performance using nasal dilators, the method comprising:
providing at least two sets of nasal dilators, wherein each set of nasal dilators has a different configuration;
providing a health quality/performance monitoring and feedback system;
receiving an identification of which set of nasal dilators of the at least two nasal dilators is used by a user;
monitoring, using the health quality/performance monitoring and feedback system, health quality/performance of the user using an identified set of nasal dilators; and
providing, using the health quality/performance monitoring and feedback system, feedback on the health quality/performance of the user using the identified set of nasal dilators.

2. The method of claim 1, wherein a second set of nasal dilators of the at least two sets of nasal dilators is provided after the health quality/performance of the user is monitored using a first set of nasal dilators.

3. The method of claim 2 wherein the second set of nasal dilators is provided based on the feedback provided on the health quality/performance of the user using the first set of nasal dilators.

4. The method of claim 1, wherein the at least two sets of nasal dilators are provided at a same time.

5. The method of claim 1, wherein a second set of the at least two sets of nasal dilators is recommended for use based on the feedback provided on the health quality/performance of the user using a first set of nasal dilators of the at least two sets of nasal dilators.

6. The method of claim 1, wherein each set of the at least two sets of nasal dilators comprises a unique identifier that is unique relative to the other set of the at least two sets of nasal dilators.

7. The method of claim 6, wherein the unique identifier comprises one or more of: a color, a number, a letter, a symbol, and an electronically readable tag.

8. The method of claim 1, wherein receiving of the identification of which set of nasal dilators of the at least two sets of nasal dilators is used by the user comprises receiving an identification of the set of nasal dilators used from the user.

9. The method of claim 8, wherein receiving an identification of the set of nasal dilators used from the user comprises receiving an input from the user on a user interface of the health quality/performance monitoring and feedback system.

10. The method of claim 9, wherein receiving an identification of the set of nasal dilators used from the user comprises scanning a unique identifier of the set of nasal dilators used.

11. The method of claim 1, wherein receiving an identification of which set of nasal dilators of the at least two set of nasal dilators is used by the user comprises providing, a recommendation of a set of nasal dilators of the at least two sets of nasal dilators.

12. The method of claim 1, wherein providing feedback comprises providing data collected during health quality/performance monitoring for the user.

13. The method of claim 1, further comprising:
providing, by the health quality/performance monitoring and feedback system, data regarding the health quality/performance of the user to a cloud computing device.

14. The method of claim 13, further comprising:
receiving, by the health quality/performance monitoring and feedback system, feedback on the health quality/performance of the user using the identified set of nasal dilators based on the data provided to the cloud computing device.

15. The method of claim 13, wherein the feedback is based on agglomerated data collected from multiple users.

16. The method of claim 13, wherein the feedback is based on health quality data from one or more other users.

17. The method of claim 1, wherein providing feedback comprises:
analyzing data collected during health quality/performance monitoring; and
providing a recommendation to the user based on analysis.

18. The method of claim 17, wherein analyzing data collected during health quality/performance monitoring is performed using artificial intelligence.

19. The method of claim 17, wherein providing a recommendation to the user based on analysis comprises recommending a nasal dilator configuration.

20. The method of claim 17, wherein analyzing data comprises combining the data from the user with data collected from other users.

21. The method of claim 17, further comprising providing the user with one or more additional nasal dilators selected or designed based on the analysis of the data collected during health quality/performance monitoring.

22. The method of claim 21, wherein the nasal dilator is provided based on data collected from other users.

23. The method of claim 21, wherein the provided one or more nasal dilators is designed based on data collected from other users.

24. The method of claim 21, wherein the provided one or more nasal dilators is designed using artificial intelligence.

25. The method of claim 21, wherein the provided one or more nasal dilators is designed for a single user.

26. The method of claim 21, wherein the provided one or more nasal dilators is designed for a group of users.

27. The method of claim 17, further comprising repeating the steps of:
receiving an identification of a nasal dilator used by the user;
monitoring, using the health quality/performance monitoring and feedback system, sleep quality of the user using the identified nasal dilator; and
providing, using the health quality/performance monitoring and feedback system, feedback on the quality of sleep using the identified nasal dilator.

28. The method of claim 1, wherein the health quality/performance monitoring and feedback system comprises a sleep monitoring and feedback system and the health quality/performance being monitored is sleep quality.

29. The method of claim 1 wherein the health quality/performance monitoring and feedback system comprises one or more of: hardware, software, a combination of hardware and software, which may be embodied in one or more discrete devices.

30. A nasal dilation system comprising:
at least two sets of nasal dilators wherein each set of nasal dilators has a different configuration; and
a health quality/performance monitoring and feedback system configured to:
monitor the health quality/performance of a user using a nasal dilator of the at least two sets of nasal dilators; and
provide feedback based on the monitored health quality/performance of the user using the nasal dilator.

31. A nasal dilation kit, comprising:
at least two sets of nasal dilators wherein each set of nasal dilators has a different configuration; and
a health quality/performance monitoring and feedback system configured to:
receive identification of which set of nasal dilators of the at least two sets of nasal dilators is used by a user;
monitor the health quality/performance of the user using an identified nasal dilator; and
provide feedback based on the monitored health quality/performance of the user using an identified nasal dilator.

32. A method of improving health quality/performance provided by nasal dilators, the method comprising:
providing one or more users at least two sets of nasal dilators wherein each set of nasal dilators has a different configuration and a health quality/performance monitoring and feedback system for each of the one or more users;
each of the one or more users:
identifying which set of nasal dilators of the at least two nasal dilators is used by the user;
monitoring, using the health quality/performance monitoring and feedback system, health quality/performance of the user using an identified set of nasal dilators; and
transmitting, using the health quality/performance monitoring and feedback system, data regarding the health quality/performance of the user;
a remote computing device:
receiving data regarding the health quality/performance of the user using the identified nasal dilator from the one or more users; and
analyzing, the received data regarding the health quality/performance from the one or more users;
modifying design parameters of a nasal dilator design based on analysis resulting from the analyzing step; and
providing to the one or more users one or more additional nasal dilators configured based on the modified design.

33. The method of claim 32, wherein the modifying of design parameters is performed by a designer.

34. The method of claim 32, wherein the providing of one or more users with one or more additional nasal dilators configured based on the modified design is performed by a provider/manufacturer.

35. The method of claim 32, wherein the analyzing of the received data is performed using artificial intelligence.

36. The method of claim 32, wherein the modifying of design parameters is data is performed using artificial intelligence.

37. The method of claim 32, wherein the one or more additional nasal dilators are configured for a specific user.

38. The method of claim 32, wherein the one or more additional nasal dilators are configured for a group of users.
